(19) United States
(12) Patent Application Publication
(10) Pub. No.: US 2026/0041681 A1
(43) Pub. Date: Feb. 12, 2026

(54) TOPICAL TREATMENT OF ARBORVIRAL INFECTIONS

(71) Applicant: Yogesh DANDIKER, Edina, MN (US)

(72) Inventor: Yogesh DANDIKER, Edina, MN (US)

(21) Appl. No.: 19/291,690

(22) Filed: Aug. 6, 2025

Related U.S. Application Data
(60) Provisional application No. 63/680,452, filed on Aug. 7, 2024.

(51) Int. Cl. A61K 31/4745; A61K 9/70; A61K 47/14; A61P 31/12

(52) U.S. Cl. CPC A61K 31/4745; A61K 9/7015; A61K 47/14; A61P 31/12

(57) ABSTRACT

The present disclosure provides a composition comprising about 0.01% w/v to about 5% w/v of resiquimod, gardiquimod, and/or their derivatives a penetration enhancer, at least 40% w/v of an aliphatic solvent, and a film forming excipient, wherein the film forming excipient is soluble at a pH between 1 and 10, wherein the composition is a solution which can be sprayed or applied using an applicator to the skin of children or adults and that forms protective and washable film when on skin and wherein the resiquimod, and gardiquimod, do not crystallize when the composition is applied to the skin surface. The disclosure provides a method of treating arboviral infections in a subject in need thereof, the method comprising topically administering to the subject the topical compositions as described herein.

### FIELD OF THE INVENTION

The present disclosure provides a composition comprising about 0.01% w/v to about 5% w/v, resquimod and/or gardiquimod, a penetration enhancer, at least 45% of an aliphatic solvent and a film forming excipient. The disclosure further provides a method of treating arboviruses, such as the Zika, Dengue and/or Chikungunya viruses in adults and children, the method comprising applying the composition as a solution or a spray to the skin.

### BACKGROUND

Arboviruses are a large, genetically diverse group of viruses transmitted by arthropods that cause a wide spectrum of diseases in humans including Zika disease virus, Dengue fever, West Nile virus disease, Yellow fever, Chikungunya virus disease, Colorado tick fever, Eastern and Western equine encephalitis, St. Louis encephalitis and Japanese encephalitis. There have been substantial increases in both the incidences and geographical ranges of arboviruses spread by mosquitoes, which infect hundreds of millions of people each year. Despite the genetic diversity in arboviruses, it is difficult to clinically differentiate between types of infections in the early stages of disease because the infection is either asymptomatic or present as a nonspecific febrile viral illness. In many geographic areas, this is compounded by the widespread co-circulation of distinct species of arboviruses in the same geographic area.

An imidazoquinolinone derivative, resiquimod, was shown to be effective in blocking Zika virus replication by inducing the antiviral protein viperine (Vanwalscappel, 2018). Gardiquimod, another imidazoquinolinone derivative, was delivered intranasally and shown to be superior to resiquimod when used as a vaccine adjuvant against Norwalk Virus (Velasquez et al., 2010).

Currently, there are no treatment or prophylaxis methods that can treat or prevent a range of viral infections are easy to use, particularly for vulnerable populations, and do not require low temperature storage.

### SUMMARY OF THE INVENTION

The present disclosure is directed to a composition comprising about 0.01% w/v to about 5% w/v resiquimod, gardiquimod and/or their derivatives, a penetration enhancer, at least 45% w/v aliphatic solvent and a film forming excipient, wherein the film forming excipient has a solubility in water at a pH between 1 and 10, and wherein the composition forms a washable and/or a peelable film when sprayed on a skin surface, and wherein the resiquimod, gardiquimod and their derivatives do not crystallize when the composition is applied to the skin surface.

In some embodiments, the disclosure provides a topical composition comprising about 0.01% w/v to about 5% w/v of resiquimod, gardiquimod and/or their derivatives, a penetration enhancer and about 45% to about 95% w/v of an aliphatic solvent.

In some embodiments, the composition comprises about 0.01% w/v to about 3% w/v resiquimod. In some embodiments, the composition comprises 0.01% w/v to about 3% w/v gardiquimod

In some embodiments, the penetration enhancer comprises 1-dodecylazacycloheptan-2-one, isopropyl myristate, octisalate, oleic acid, diethylene glycol monoethyl ether (Transcutol® P), or combinations thereof. In some embodiments, the penetration enhancer is octisalate. In some embodiments, the penetration enhancer is about 1% to about 15% w/v of the composition.

In some embodiments, the solvent comprises acetone, di-isopropyl adipate, dimethyl isosorbide, dimethyl sulfoxide, ethyl acetate, ethanol, isopropyl alcohol or combinations thereof. In some embodiments, the solvent is ethanol, isopropyl alcohol or combinations thereof.

In some embodiments, the composition comprises about 3% w/v to about 7% w/v of a film forming excipient. In some embodiments, the film forming excipient comprises a polyacrylate polymer or a cellulose polymer. In some embodiments, the film forming excipient is methacrylic acid and methyl methacrylate copolymer 1:1, methacrylic acid and methyl methacrylate copolymer 1:2, poly (butylmethacrylate-co-(2-dimethylaminoethyl) methacrylate-co-methyl methacrylate 1:2:1, hypromellose, hydroxypropyl cellulose, and ethyl cellulose.

In some embodiments, the penetration enhancer is octisalate, and the film forming excipient is methacrylic acid and methyl methacrylate copolymer 1:1 or the film forming excipient is poly (butyl methacrylate-co-(2-dimethylaminoethyl) methacrylate co-methyl methacrylate 1:2:1).

In some embodiments, the composition further comprises a washability enhancer. In some embodiments, the washability enhancer is polyethylene glycol 400. In some embodiments, the composition further comprises tromethamine.

In some embodiments, the pH of the composition is about 4 to about 5. In some embodiments, the pH of the composition is about 4.5. In some embodiments, the composition forms a barrier film in less than five minutes after application to the skin. In some embodiments, the composition forms a barrier film less than three minutes after application to the skin surface. In some embodiments, the composition forms a barrier film less than one minute after application to the skin. In some embodiments, the barrier film formed is water washable. In some embodiments, the barrier film is capable of preventing transfer of resiquimod and/or gardiquimod to another human.

In some embodiments, the composition has a viscosity of less than 30 cPs at room temperature. In some embodiments, the composition has a water vapor transmission rate, as a fraction compared to a non-occluded control, not less than 0.50 over a 48-hour period when applied and dried on a porous substrate. In some embodiments, the composition is suitable for application as a spray, transdermal patch, gel, cream, spray or lotion

In some embodiments, the present disclosure provides a spray container comprising the compositions described herein, and a metering valve. In some embodiments, the container further comprises an applicator for spreading the composition onto human skin. In some embodiments, the container further comprises a dose indicator. In some embodiments, the container is pressurized. In some embodiments, the container further comprises a propellent.

In some embodiments, the present disclosure provides a method of treating arboviral infections comprising topically applying the any one of the compositions disclosed herein to a skin surface of the subject. In some embodiments, the present disclosure provides a method of method of increasing interferon levels comprising topically applying the any one of the compositions disclosed herein to a skin surface of the subject. In some embodiments, the arboviral infection is a Zika, Dengue or Chikungunya viral infection. In some embodiments, the composition is applied as a spray.

In some embodiments, the composition is applied at least as a first application at a first timepoint and a second application at a second timepoint, wherein the second timepoint is at least 24 hours after the first timepoint.

In some embodiments, the composition is applied at three or more applications at three or more timepoints. In some embodiments, the time between each timepoint is about 24 hours.

In some embodiments, the first application and the second application are applied at the same site on the skin surface of the subject. In some embodiments, the first application and the second application are applied at different sites on the skin surface of the subject. In some embodiments, the barrier film formed from the first application is removed before applying the second application. In some embodiments, the barrier film formed from the previous application is removed before applying the subsequent application.

In some embodiments, the subject is human.

In some embodiments, the composition is applied to the skin surface in a single actuation in a volume of about 500 μL or less. In some embodiments, the composition is applied to the skin surface in a single actuation in a volume of about 250 μL or less. In some embodiments, the composition is applied to the skin surface in a single actuation in a volume of about is about 100 μL or less. In some embodiments, the composition is applied to the skin surface in a single actuation in a volume of about 50 μL to about 300 μL.

### BRIEF DESCRIPTION OF THE FIGURES

FIG. 1 is a graph illustrating the skin permeation profiles of a 0.01% w/v of resiquimod and 0.01 w/v of gardiquimod film forming composition as outlined in Example 1.

### DETAILED DESCRIPTION OF THE INVENTION

The present disclosure relates to topical compositions comprising resiquimod, gardiquimod and/or their derivatives and methods suitable for the treatment arboviral infections. The present disclosure provides for a new composition which is a sprayable liquid, which in some embodiments can form a film on the skin after the spray has dried. The film can act as barrier that prevents transfer of resiquimod, gardiquimod and/or their derivatives from the application site to other individuals, e.g., the user's partner or individuals in close proximity. Additionally, the film can be washable as it can conveniently be removed with water while performing daily activities such as a shower. In some embodiments, the sprayable liquid compositions described herein can be sprayed directly on the skin surface, and do not need to be spread over the skin by touching with fingers or with use of a separate applicator. In some embodiments, drug absorption into the skin is rapid. In some embodiments drug absorption from the sprayable liquid composition into the skin is complete when the protective film has dried (<5 mins). In some embodiments, the film can be washed off once it has dried. In some embodiments, during solvent evaporation, the excipients included in the sprayable liquid compositions can prevent recrystallization of resiquimod, gardiquimod and/or their derivatives, a feature that can be important for its penetration of the skin. In some embodiments, the resiquimod, gardiquimod and/or their derivatives in the composition described herein do not crystallize when the composition is applied to the skin. In some embodiments, the protective film is breathable which prevents erythema of the skin from developing over long-term use. In some embodiments, direct application to the skin via a sprayable liquid composition provides for increased convenience, and lower transmission of the resiquimod, gardiquimod and/or their derivatives to the hands/fingers or other body parts for which it is not needed. In some embodiments, the sprayable liquid compositions described herein can be applied quickly. In some embodiments, the sprayable liquid compositions described herein can be applied quickly over a large area of the skin.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. A used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “room temperature” is an indoor temperature suitable for long term storage of biological matter and laboratory experimentation, typically ranging between 15-28° C. In embodiments, room temperature is from 20-25° C.

Imidazoquinolinone derivatives possess antiviral properties and thus, have been developed and tested in preclinical studies and clinical trials for the treatment of viral infections. Prominent compounds in this class of compounds are resiquimod (also known as R848), and gardiquimod as shown below, both of which have been shown to be 100 times more potent than the approved imidazoquinolinone, imiquimod, in stimulating Toll Like Receptors (TLR) with antiviral and antitumor activity.

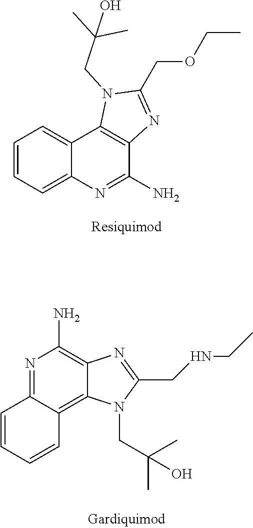 [SMILES: CCOCc1nc2c(N)nc3ccccc3c2n1CC(C)(C)O ; CCNCc1nc2c(N)nc3ccccc3c2n1CC(C)(C)O]

The present disclosure is directed to a composition comprising about 0.01% w/v to about 5% w/v of resiquimod, gardiquimod and/or their derivatives, a penetration enhancer, at least 45% w/v of an aliphatic solvent and a film forming excipient, wherein the film forming excipient has a solubility in water, at a pH between 1 and 10, wherein the film forming excipient has a solubility in water at a pH between 1 and 10, wherein the composition forms a washable and/or a peelable film when sprayed on a skin surface, and wherein the resiquimod, gardiquimod and their derivatives do not crystallize when the composition is applied to the skin surface.

The present disclosure is directed to a topical composition comprising about 0.01% w/v to about 5% w/v of resiquimod, gardiquimod and/or their derivatives, a penetration enhancer, and about 45% to about 95% w/v of an aliphatic solvent.

Various concentrations of resiquimod, gardiquimod and/or their derivatives can be used in the composition. In some embodiments, the composition comprises about 1% w/v to about 25% w/v resiquimod, gardiquimod and/or their derivatives. In some embodiments, the composition comprises about 1% w/v to about 20% w/v resiquimod, gardiquimod and/or their derivatives. In some embodiments, the composition comprises about 1% w/v to about 19% w/v resiquimod, gardiquimod and/or their derivatives. In some embodiments, the composition comprises about 1% w/v to about 18% w/v resiquimod, gardiquimod and/or their derivatives. In some embodiments, the composition comprises about 1% w/v to about 17% w/v resiquimod, gardiquimod and/or their derivatives. In some embodiments, the composition comprises about 1% w/v to about 16% w/v resiquimod, gardiquimod and/or their derivatives. In some embodiments, the composition comprises about 1% w/v to about 15% w/v resiquimod, gardiquimod and/or their derivatives. In some embodiments, the composition comprises about 1% w/v to about 14% w/v resiquimod, gardiquimod and/or their derivatives. In some embodiments, the composition comprises about 1% w/v to about 13% w/v resiquimod, gardiquimod and/or their derivatives. In some embodiments, the composition comprises about 1% w/v to about 12% w/v resiquimod, gardiquimod and/or their derivatives. In some embodiments, the composition comprises about 1% w/v to about 11% w/v resiquimod, gardiquimod and/or their derivatives. In some embodiments, the composition comprises about 1% w/v to about 10% w/v resiquimod, gardiquimod and/or their derivatives. In some embodiments, the composition comprises about 1% w/v to about 9% w/v resiquimod, gardiquimod and/or their derivatives. In some embodiments, the composition comprises about 1% w/v to about 8% w/v resiquimod, gardiquimod and/or their derivatives. In some embodiments, the composition comprises about 1% w/v to about 7% w/v resiquimod, gardiquimod and/or their derivatives. In some embodiments, the composition comprises about 1% w/v to about 6% w/v resiquimod, gardiquimod and/or their derivatives. In some embodiments, the composition comprises about 1% w/v to about 5% w/v resiquimod, gardiquimod and/or their derivatives. In some embodiments, the composition comprises about 1% w/v to about 4% w/v resiquimod, gardiquimod and/or their derivatives. In some embodiments, the composition comprises about 1% w/v to about 3% w/v resiquimod, gardiquimod and/or their derivatives.

Various aliphatic solvents can be used in the sprayable liquid compositions of the present disclosure. The term “aliphatic solvent” enhancer refers to any solvent comprising compounds that are without a ring structure, e.g., without an aromatic ring structure. In some embodiments, the term “aliphatic solvent” refers to mixtures of saturated, long straight-chain, branched-chain, or cyclic paraffins that have low viscosity, e.g., less than 500 cP, less than 100 cP, less than 50 cP, less than 30 cP, less than 10 cP, or less than 5 cP at 20° C., which are suitable for being applied by spraying. In some embodiments, the aliphatic solvent is suitable for solubilizing the film forming excipients. In some embodiments, the aliphatic solvent is suitable for solubilizing resiquimod, gardiquimod and/or their derivatives, e.g., a non-aromatic solvent, which is pharmaceutically acceptable. In some embodiments, the aliphatic solvent is suitable for solubilizing both the film forming excipients and resiquimod or gardiquimod. In some embodiments, the solvent is an alcoholic aliphatic solvent. In some embodiments, the aliphatic solvent is ethylene, isooctane, acetylene, propene, propane, squalene, acetone, ethanol, methanol, propanol, butanol, isopropyl alcohol, di-isopropyl adipate, di-methyl sulphoxide, ethyl acetate, and polyethylene. In some embodiments, aliphatic solvents can include more than one solvent, e.g., a mixture of aliphatic solvents. In some embodiments, the solvent is ethanol or a mixture of ethanol with other aliphatic solvents. In some embodiments, the solvent comprises ethyl acetate, ethanol, isopropyl alcohol, or combinations thereof. In some embodiments, the aliphatic solvent is a combination of ethyl acetate, ethanol, and isopropyl alcohol. In some embodiments, the aliphatic solvent has a viscosity suitable for administering via an aerosol spray or a mist. In some embodiments, the aliphatic solvent can be used to solubilize the sprayable liquid compositions described herein, i.e., the sprayable liquid compositions are a solution.

In some embodiments, the aliphatic solvent comprises two solvents at a ratio of about 1:100 to about 100:1. In some embodiments, the aliphatic solvent comprises two solvents at a ratio of about 5:95 to about 95:5. In some embodiments, the aliphatic solvent comprises two solvents at a ratio of about 20:80 to about 80:20. In some embodiments, the aliphatic solvent comprises two solvents at ratio of about 30:70 to about 70:30. In some embodiments, the aliphatic solvent comprises two solvents at a ratio of about 40:60 to about 60:40. In some embodiments, the aliphatic solvent comprises two solvents at a ratio of about 50:50.

In some embodiments, the aliphatic solvent comprises three solvents at a ratio of about 1:1:1 to about 1:1:10. In some embodiments, the aliphatic solvent comprises three solvents at a ratio of about 1:2:2 to about 1:2:4. In some embodiments, the aliphatic solvent comprises three solvents at a ratio of about 1:3:1 to about 1:3:6. In some embodiments, the aliphatic solvent comprises three solvents at a ratio of about 1:4:1 to about 1:4:12.

The total aliphatic solvent concentration can be around 30% to about 97% weight of the composition, but in some embodiments the aliphatic solvent is in a sufficient quantity to dissolve the other excipients, resiquimod, gardiquimod and/or their derivatives. In some embodiments, the composition comprises about 65% weight aliphatic solvent. In some embodiments, the composition comprises about 50% to about 90% by weight aliphatic solvent. In some embodiments, the composition comprises about 30% to about 90%, about 40% to about 90%, about 60% to about 90% or about 70% to about 90% by weight aliphatic solvent. In some embodiments, the composition comprises about 52% to about 68%, about 54% to about 66%, about 56% to about 64% or about 58% to about 62% by weight aliphatic solvent. In some embodiments, the composition comprises about 35% to about 45% by weight aliphatic solvent. In some embodiments, the composition comprises about 70% by weight aliphatic solvent. In some embodiments, the composition comprises less than 80%, less than 70%, less than 65%, less than 62% or less than 61% by weight of aliphatic solvent. In some embodiments, the composition comprises about 70% to about 85% alcoholic solvent, about 72% to about 82% aliphatic solvent, or about 74% to about 80% aliphatic solvent by weight. In some embodiments, the composition comprises about 75%, about 76%, about 77%, about 78%, about 79%, about 80%, about 81%, about 82% or about 83% aliphatic solvent by weight. In some embodiments, the reduced aliphatic solvent concentration can result in reduced adverse effect, e.g., inflammation or irritation.

In some embodiments, the aliphatic solvent comprises acetone, di-isopropyl adipate, dimethyl isosorbide, dimethyl sulfoxide, ethyl acetate, ethanol, isopropyl alcohol or combinations thereof. In some embodiments, the aliphatic solvent comprises ethanol, isopropyl alcohol or combinations thereof. In some embodiments, the ethanol and isopropyl alcohol are in a ratio of about 1:1:1 to about 1:1:10. In some embodiments, the ethanol and isopropyl alcohol are in a ratio of about 1:2:2 to about 1:2:4. In some embodiments, the ethanol and isopropyl alcohol are in a ratio of about 1:3:1 to about 1:3:6. In some embodiments, the ethanol and isopropyl alcohol are in a ratio of about 1:4:1 to about 1:4:12.

In some embodiments, when the liquid sprayable composition is applied to the skin, the solvent is volatile and evaporates (i.e., dries), leaving a trace amount (or less) of the solvent on the skin. In some embodiments, the aliphatic solvent is a volatile solvent. For example, in some embodiments, the solvent evaporates, i.e., 500 μL, 250 μL, or 100 μL vaporizes in less than two minutes, in less than one minute or in less than 30 seconds after the composition is sprayed onto a surface at room temperature and 1 atmospheric pressure.

The present disclosure provides for resiquimod or gardiquimod sprayable liquid compositions comprising a film forming excipient. In some embodiments, the disclosure provides for compositions, e.g., topical spray compositions comprising a film forming excipient, wherein the film forming excipient comprises a polyacrylate polymer or a cellulose polymer. In some embodiments, such compositions provide for uniform drug distribution and dose, increased bioavailability, continuous drug release and longer-lasting effect to the treated area, while minimizing transfer of resiquimod or gardiquimod. In some embodiments, the long-lasting effect of the present compositions allow for a reduced number of total administrations of the resiquimod or gardiquimod composition. In some embodiments, the long-lasting effect of the present compositions allow for a reduced frequency of administration of the resiquimod or gardiquimod composition.

The film forming excipient is an excipient, preferably a polymer, that is soluble in aliphatic solvents, preferably in ethanol or a mixture of ethanol with other solvents. The film forming excipient is also soluble in aqueous solutions, preferably water. Although, for film forming excipients having pH dependent solubility, the pH of the aqueous solution must be above or below the specific trigger pH for that excipient to dissolve. For example, some of these film forming excipients dissolve in aqueous solutions only above pH 6.0, only above pH 7.0, or only below pH 5.0. In some embodiments, the film forming excipient has a solubility in water, at a pH between 1 and 10.

In some embodiments, the sprayable liquid composition comprises a film forming excipient comprising a polyacrylate polymer or a cellulose polymer. Polyacrylate polymers are commercially available and known to those in the art, and can refer to a group of polymers prepared from acrylate monomers. Polyacrylate polymers can include methacrylic acid and methyl methacrylate copolymer 1:1, methacrylic acid and methyl methacrylate copolymer 1:2, and poly(butylmethacrylate-co-(2-dimethylaminoethyl) methacrylate-co-methyl methacrylate 1:2:1. In some embodiments, the film forming excipient is butyl methacrylate and methyl copolymer (3:1), polyvinylpyrrolidone or combinations thereof. The term cellulose polymer refers to a group of polymers prepared from glucose monomers. In some embodiments, the film forming excipients used herein can exhibit properties that are important for topical compositions including high tensile strength, lack of deformity under minimal tensile stress, and low sensitivity to microbial contamination. In some embodiments, the film forming excipient allows for penetration of resiquimod or gardiquimod into the skin. In some embodiments, the film forming excipient allows moisture vapor to pass through the skin.

In some embodiments, the film forming excipient is a polyacrylate polymer. In some embodiments, the polyacrylate polymer is methacrylic acid and methyl methacrylate copolymer. In some embodiments, the methacrylic acid and methacrylate copolymer are in a ratio of about 1:4 to about 4:1, about 1:3 to about 3:1, about 1:2 to about 2:1. In some embodiments, the methacrylic acid and methacrylate copolymer are in ratio of 1:1. In some embodiments, the methacrylic acid and methacrylate copolymer are in ratio of 1:2. In some embodiments the polyacrylate polymer is methacrylic acid and methyl methacrylate copolymer 1:2. In some embodiments the polyacrylate polymer is poly(butylmethacrylate-co-(2-dimethylaminoethyl) methacrylate-co-methyl methacrylate 1:2:1. Various cellulose polymers are known in the art, and can include hypromellose, hydroxypropyl cellulose, and ethyl cellulose.

In some embodiments, the cellulose polymer is hypromellose. In some embodiments the cellulose polymer is hydroxypropyl cellulose. In some embodiments the cellulose polymer is ethyl cellulose. In some embodiments, the sprayable liquid compositions can have various combinations of penetration enhancers and film forming excipients. In some embodiments, the composition comprises the penetration enhancer octisalate, and the film forming excipient methacrylic acid and methyl methacrylate copolymer 1:1.

In some embodiments, the sprayable liquid composition can have more than one film forming excipient. In some embodiments, the composition comprises about 1% w/v to about 10% w/v film forming excipient. In some embodiments, the composition comprises about 1% w/v to about 9% w/v film forming excipient. In some embodiments, the composition comprises about 1% w/v to about 8% w/v film forming excipient. In some embodiments, the composition comprises about 1% w/v to about 7% w/v film forming excipient. In some embodiments, the composition comprises about 1% w/v to about 6% w/v film forming excipient. In some embodiments, the composition comprises about 1% w/v to about 5% w/v film forming excipient. In some embodiments, the composition comprises about 1% w/v to about 4% w/v film forming excipient. In some embodiments, the composition comprises about 1% w/v to about 3% w/v film forming excipient. In some embodiments, the composition comprises about 1% w/v to about 2% w/v film forming excipient.

In some embodiments, the sprayable liquid composition comprises about 2% w/v to about 10% w/v film forming excipient. In some embodiments, the composition comprises about 2% w/v to about 9% w/v film forming excipient. In some embodiments, the composition comprises about 2% w/v to about 8% w/v film forming excipient. In some embodiments, the composition comprises about 2% w/v to about 7% w/v film forming excipient. In some embodiments, the composition comprises about 2% w/v to about 6% w/v film forming excipient. In some embodiments, the composition comprises about 2% w/v to about 5% w/v film forming excipient. In some embodiments, the composition comprises about 2% w/v to about 4% w/v film forming excipient. In some embodiments, the composition comprises about 2% w/v to about 3% w/v film forming excipient.

In some embodiments, the sprayable liquid composition comprises about 3% w/v to about 10% w/v film forming excipient. In some embodiments, the composition comprises about 3% w/v to about 9% w/v film forming excipient. In some embodiments, the composition comprises about 3% w/v to about 8% w/v film forming excipient. In some embodiments, the composition comprises about 3% w/v to about 7% w/v film forming excipient. In some embodiments, the composition comprises about 3% w/v to about 6% w/v film forming excipient. In some embodiments, the composition comprises about 3% w/v to about 5% w/v film forming excipient. In some embodiments, the composition comprises about 3% w/v to about 4% w/v film forming excipient.

In some embodiments, the sprayable liquid composition comprises about 4% w/v to about 10% w/v film forming excipient. In some embodiments, the composition comprises about 4% w/v to about 9% w/v film forming excipient. In some embodiments, the composition comprises about 4% w/v to about 8% w/v film forming excipient. In some embodiments, the composition comprises about 4% w/v to about 7% w/v film forming excipient. In some embodiments, the composition comprises about 4% w/v to about 6% w/v film forming excipient. In some embodiments, the composition comprises about 4% w/v to about 5% w/v film forming excipient.

In some embodiments, the sprayable liquid composition comprises about 5% w/v to about 10% w/v film forming excipient. In some embodiments, the composition comprises about 5% w/v to about 9% w/v film forming excipient. In some embodiments, the composition comprises about 5% w/v to about 8% w/v film forming excipient. In some embodiments, the composition comprises about 5% w/v to about 7% w/v film forming excipient. In some embodiments, the composition comprises about 5% w/v to about 6% w/v film forming excipient.

In some embodiments, the sprayable liquid composition comprises about 6% w/v to about 10% w/v film forming excipient. In some embodiments, the composition comprises about 6% w/v to about 9% w/v film forming excipient. In some embodiments, the composition comprises about 6% w/v to about 8% w/v film forming excipient. In some embodiments, the composition comprises about 6% w/v to about 7% w/v film forming excipient. In some embodiments, the composition comprises about 7% w/v to about 10% w/v film forming excipient. In some embodiments, the composition comprises about 7% w/v to about 9% w/v film forming excipient. In some embodiments, the composition comprises about 7% w/v to about 8% w/v film forming excipient. In some embodiments, the composition comprises about 8% w/v to about 10% w/v film forming excipient. In some embodiments, the composition comprises about 8% w/v to about 9% w/v film forming excipient. In some embodiments, the composition comprises about 9% w/v to about 10% w/v film forming excipient.

In some embodiments, the composition comprises about 5% w/v to about 7% w/v film forming excipient. In some embodiments, the composition comprises about 5% w/v to about 7% w/v film forming excipient. In some embodiments, the composition comprises about 5% w/v to about 7% w/v film forming excipient. In some embodiments, the composition comprises about 6% w/v to about 7% w/v film forming excipient.

In some embodiments, the sprayable liquid composition can comprise a penetration enhancer. The term “penetration enhancer” refers to any compound or composition that penetrate into the skin and interact with skin constituents to promote drug flux or reversibly decrease the barrier resistance. Penetration enhancers can include substances which promote the dermal absorption of resiquimod or gardiquimod through the skin temporarily by transiently disturbing the lipid bilayer structure in the stratum corneum barrier or enhancing the solubility of the drug in the skin to facilitate drug delivery. In some embodiments, the penetration enhancer can include any penetration enhancer known to be pharmaceutically acceptable. In some embodiments, the penetration enhancer can include any skin enhancer as found in the U.S. Pharmacopeia, or otherwise known in the art, e.g., M.E. Lane, Skin Permeation Enhancers, Int. J. Pharma 447(1-2):12-21 (2013). In some embodiments, the penetration enhancer can include 1-dodecylazacycloheptan-2-one, isopropyl myristate, octisalate, oleic acid, diethylene glycol monoethyl ether (Transcutol®), or combination thereof. In some embodiments, one, two, three or greater than three penetration enhancers are on the sprayable liquid composition. In some embodiments, two penetration enhancers are in the composition. In some embodiments, the penetration enhancer is azone, isopropyl myristate, octisalate, oleic acid, Transcutol® P or combinations thereof. In some embodiments, the penetration enhancer is octisalate.

Various concentrations of penetration enhancers can be present in the sprayable liquid composition. The permeation enhancer can be adjusted to facilitate the penetration of resiquimod or gardiquimod. In some embodiments, the composition comprises about 0.1% w/v to about 20% w/v, from about 0.1% w/v to about 15% w/v, from about 0.1% w/v to about 10% w/v, about 0.1% w/v to about 5% w/v, or about 0.5% w/v to about 8% w/v penetration enhancer. In some embodiments, the composition comprises about 4% to about 25% by weight penetration enhancer. In some embodiments, the composition comprises about 4% to about 20% by weight penetration enhancer. In some embodiments, the composition comprises about 4% to about 15% by weight penetration enhancer. In some embodiments, the composition comprises about 4% to about 10% by weight penetration enhancer. In some embodiments, the composition comprises about 10% to about 15% by weight penetration enhancer. In some embodiments, the composition comprises about 10% to about 20% by weight penetration enhancer. In some embodiments, the composition comprises about 15% to about 20% by weight penetration enhancer. In some embodiments, the composition comprises about 15% to about 25% by weight penetration enhancer.

In some embodiments, the sprayable liquid composition comprises about 1% to about 10% of the penetration enhancer. In some embodiments, the composition comprises about 2% to about 10% of the penetration enhancer. In some embodiments, the composition comprises about 3% to about 10% of the penetration enhancer. In some embodiments, the composition comprises about 4% to about 10% of the penetration enhancer. In some embodiments, the composition comprises about 5% to about 10% of the penetration enhancer. In some embodiments, the composition comprises about 6% to about 10% of the penetration enhancer. In some embodiments, the composition comprises about 7% to about 10% of the penetration enhancer. In some embodiments, the composition comprises about 8% to about 10% of the penetration enhancer. In some embodiments, the composition comprises about 9% to about 10% of the penetration enhancer.

In some embodiments, the film forming excipient is butyl methacrylate and methyl copolymer (3:1), or combinations thereof, and the penetration enhancer is octisalate.

In some embodiments, the sprayable liquid compositions comprising the film forming excipients herein form a non-tacky, flexible, adhesive and peelable film, or otherwise removable solid that is coherent as a thin solid layer when dried. As used herein, the term “peelable” refers to materials that can be removably applied to and adhere to surfaces such as the surface of mammalian and human skin and can be subsequently removed from the skin by physical force. Such peelable materials can be effective to exfoliate skin and remove comedones by mechanical means. Peelable films according to the compositions and methods of this invention can be adhesively and removably applied to human skin and, by virtue of being forcibly removed, carry away the dead skin cells, hair, dirt, sebum and blackheads which have adhered to the films while leaving behind the skin benefit agents and active ingredients on the skin. In some embodiments, the peelable films of this invention have an adhesive force of at least about 5 to about 25 ounces (oz). In some embodiments, the peelable films of this invention do not have an adhesive force that cause disruption to the outer layer of the skin and thereby damage the skin. In some embodiments, the adhesive force of the peelable films of this invention are sufficiently high to enable the films to exfoliate and remove comedones from the skin. In some embodiments, the cohesive film can be peelable from the skin, e.g., the cohesive film can remain as a single large piece when peeled from the skin, or tears into two or three large pieces. In some embodiments, the peelable film removes undesirable materials from the skin, for example excessive dead skin cells and comedones.

In some embodiments, the sprayable liquid composition forms a washable film. In some embodiments, the present disclosure provides sprayable liquid compositions that can be washed off via convenient methods, i.e., showering, when absorption of the drug into the skin is complete. In some embodiments, washability includes removal of the film without the use of a detergent. The ability to easily wash off the dried film in the composition allows the user to reapply the subsequent dose of the composition at the previous site of application. A washability enhancer can be included in the composition to make the film more washable, i.e., easier to remove from skin with water or soap and water, particularly when the film forming excipient is an excipient that requires a certain pH to dissolve in water or an aqueous environment, such as methacrylic acid-methyl methacrylate copolymer (1:2) (Eudragit® S100). In some cases, the washability enhancer also acts as a plasticizer. Moreover, the film forming excipient, or mixture of film forming excipient with a plasticizer and/or washability enhancer, prevents recrystallization of resiquimod or gardiquimod and maintains resiquimod or gardiquimod in an amorphous state during and after solvent evaporation. In some embodiments, the washability enhancer is a low molecular weight polyethylene glycol (PEG) containing ethylene glycol polymer of various molecular weights, such as polyethylene glycol 300, 400 or 600.

The concentration of washability enhancer in the sprayable liquid composition can be about 1% to about 300% of the weight of the film forming excipient in the composition. In some embodiments it is about 50% to about 250% of the weight of the film forming excipient in the composition. In other embodiments, it is about 20% to about 100% of the weight of the film forming excipient in the composition. It can also be is about 40%, about 45%, about 50%, about 55%, about 60%, about 70%, about 80%, about 90%, about 100%, about 120%, about 140%, about 160%, about 180%, about 190%, about 200%, about 210%, about 220%, about 230%, about 240%, or about 250% of the weight of the film forming excipient in the composition. With regard to the total weight of the composition, in some embodiments, the concentration of washability enhancer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition.

The sprayable liquid composition can include one or more plasticizers such as dibutyl sebacate, triethyl citrate, triacetin, glycerol, a low molecular weight polyethylene glycol, e.g., polyethylene glycol 300, 400 or 600, and/or propylene glycol. The concentration of plasticizer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition. In some embodiments it is about 0.5% w/v, about 1% w/v, about 1.5% w/v, about 2% w/v, about 2.5% w/v, about 3% w/v, about 3.5% w/v, or about 4% w/v, of the composition. In some embodiments, the plasticizer is also a washability enhancer.

The sprayable liquid composition can also include one or more viscosity increasing agents, e.g., povidone, glycerin, hydroxypropyl cellulose, methylcellulose, and/or carboxymethylcellulose. The concentration of the viscosity increasing agent, if included, can be from about 0.1% w/v to about 10% w/v, about 0.1% w/v to about 8% w/v, about 0.1% w/v to about 5% w/v of the composition, e.g., about 0.5% w/v, about 1% w/v, about 1.5% w/v, about 2% w/v, or about 2.5% w/v of the composition. In some embodiments, the viscosity increasing agent is in an amount that allows the composition to still be a sprayable liquid solution. For example, in some embodiments, the viscosity of the composition is less than 40 mPas, less than 30 mPas, less than 20 mPas, less than 10 mPas or less than 5 mPas at room temperature and 1 atmospheric pressure.

In some embodiments, the sprayable liquid compositions can comprise viscosity increasing agents. In some embodiments, the viscosity increasing agent is a bioadhesive. Such agents include Carbopol®. Carbopol® polymers are high molecular weight, crosslinked polyacrylic acid polymers. The polymers differ by crosslink density and can be grouped into the following categories: (1) Carbopol® homopolymers, such as acrylic acid crosslinked with allyl sucrose or allyl pentaerythritol, e.g., Carbopol® 71G NF (viscosity 4,000-11,000), 971P NF (viscosity 4,000-11,000), 974P NF, (viscosity 29,400-39,400), 980 NF (viscosity 40,000-60,000), 981 NF (viscosity 4000-10,000), 5984 EP (viscosity 30,500-39,400), 934 NF (viscosity 30,500-39,400), 934P NF (viscosity 29,400-39,400), 940 NF (viscosity 40,000-60,000), 941 NF (viscosity 4,000-10,000); and (2) Carbopol® copolymers: acrylic acid and C10-C30 alkyl acrylate crosslinked with allyl pentaerythritol, e.g., Carbopol® 1342 NF, viscosity 9,500-26,500.

The compositions of the present disclosure can also include other excipients, added, e.g., to achieve a desired consistency or appearance, or to protect the composition components from degradation and oxidation. Such excipients include, for example, cosolvents, stabilizing agents, antioxidants, humectants, preservatives, pH modifiers, colorants, dyes, and fragrances known in the art of formulation.

Another optional excipient of the compositions is a pH modifier, such as a buffer. In some embodiments, the buffer comprises sodium citrate, citric acid, fumaric acid, monopotassium phosphate, boric acid, acetic acid, adipic acid, calcium hydroxide, glycine, hydrochloric acid, lactic acid, magnesium aluminometasilicates, phosphoric acid, sodium carbonate, sodium hydroxide, sorbic acid, succinic acid, tartaric acid, and derivatives, salts, diethyl barbituric acid or combinations thereof. In some embodiments, the buffer is present in the composition at a concentration of is present in the composition at a concentration from 1 mM to 100 mM, about 1 mM to 50 mM, about 5 mM to 25 mM or about 10 mM. In some embodiments, the buffer is present in the composition at a concentration of 100 mM. In some embodiments, the pH of the sprayable liquid composition is about 3 to about 6, about 3.5 to about 5.5, or about 4 to about 5. In some embodiments, the pH of the sprayable liquid composition is about 4.5.

In some embodiments, the addition of emollients, emulsion stabilizers, moisturizers, excipients, and other compounds may be modified to enhance the sensory properties of the sprayable topical compositions, including but not limited to the skin feel (silkiness, lightness, creaminess, etc.), soften, soothe and moisturize the skin, support renewal of post-procedure skin, help restore the skin's moisture balance, hydrate dry and compromised skin, and is suitable for extremely dry skin. In some embodiments, the composition comprises a moisturizer. In some embodiments, the moisturizer is petrolatum, microcrystalline wax, physalis angulata extract, caprylic/capric triglyceride, Butyrospermum parki (shea butter) extract, bisabolol, and tocopherol.

In some embodiments, particularly for application to the skin, the composition can comprise a fragrance or perfume to impart a desired aroma, or to mask odors that can be associated with other components of the composition. In some embodiments, the concentration of the fragrance is about 0.01% w/v to about 5% w/v of the composition, or about 0.1% w/v to about 1% w/v of the composition.

Any fragrance suitable for application to the skin can be used herein including a wide variety of fragrances and perfumes that are known to those skilled in the art. In some embodiments, the amount of fragrance can be effective for providing a noticeable aroma to the composition, or for masking undesired aroma of the composition. In some embodiments, the fragrance does not impart excessive stinging to the skin, especially to broken or irritated skin.

Typical fragrances are described in Arctander, Perfume and Flavour Chemicals (Aroma Chemicals), Vol. I and II (1969, Allured Publishing Corporation, 1969 (ISBN 0931710375, 9780931710377), and Arctander, Perfume and Flavor Materials of Natural Origin (1994, by Allured Pub Corp) (ISBN 0931710367; ISBN13: 9780931710360). Fragrance used in the present composition can also contain solubilizers, diluents, or solvents which are well known in the art.

In some embodiments, application of resiquimod or gardiquimod compositions without a protective cover or barrier over the area can leave unabsorbed drug exposed, which creates a risk of transfer of resiquimod or gardiquimod to other persons or surfaces and unwanted side effects. Additionally, in some embodiments, the loss of resiquimod or gardiquimod after transfer from the user's skin to other surfaces can also alter the amount of the drug remaining on the skin necessary for its intended therapeutic effect. The present disclosure provides a sprayable liquid composition that prevents transfer of resiquimod or gardiquimod.

In some embodiments, the barrier film is occlusive. In some embodiments, the occlusive film is transparent. In some embodiments, occlusive refers to a film that forms a protective layer on a surface, meaning the film prevents transference of resiquimod or gardiquimod to other humans. In some embodiments, the occlusive film forms a protective layer against bacteria and viruses. In some embodiments, the occlusive film is breathable and allows moisture vapor to be able to pass through the film, which maintains the integrity of the skin.

As the barrier film prevents transfer of resiquimod or gardiquimod, it is important for the sprayable liquid composition to quickly dry after application and form the barrier film. In some embodiments, the sprayable liquid composition dries quickly after application. For example, in some embodiments, the sprayable liquid composition dries within 90 seconds, within 80 seconds, within 70 seconds or within 60 seconds after application. In some embodiments, the sprayable liquid composition forms a barrier film less than three minutes after application to the skin. In some embodiments, the sprayable liquid composition forms a barrier film less than two minutes after application to the skin. In some embodiments, the sprayable liquid composition forms a barrier film in less than one minute after application to the skin. For example, in some embodiments, the sprayable liquid composition forms a barrier film within 90 seconds, within 80 seconds, within 70 seconds or within 60 seconds after application.

In some embodiments, the sprayable liquid composition dries quickly after application. For example, in some embodiments, the sprayable liquid composition dries within 90 seconds, within 80 seconds, within 70 seconds or within 60 seconds after application of 500 μL, 250 μL, 100 μL or 50 μL of the composition to the skin at room temperature and 1 atmospheric pressure. In some embodiments, the sprayable liquid composition forms a barrier film less than three minutes after application of 500 μL, 250 μL, 100 μL or 50 μL of the composition to the skin at room temperature and 1 atmospheric pressure. In some embodiments, the sprayable liquid composition forms a barrier film less than two minutes after application of 500 μL, 250 μL, 100 μL or 50 μL of the composition to the skin at room temperature and 1 atmospheric pressure. In some embodiments, the sprayable liquid composition forms a barrier film in less than one minute after application of 500 μL, 250 μL, 100 μL or 50 μL of the composition to the skin at room temperature and 1 atmospheric pressure. For example, in some embodiments, the sprayable liquid composition forms a barrier film within 90 seconds, within 80 seconds, within 70 seconds or within 60 seconds after application of 500 μL, 250 μL, 100 μL or 50 μL of the composition at room temperature and 1 atmospheric pressure.

In some embodiments, the use of transdermal patches or topical solutions with a protective cover or barrier over the area can result in skin sensitization and/or irritation, discomfort from adhesives, and imperfect skin adhesion. The present disclosure provides compositions that are designed to remain intact for an extended period of time, e.g., for 24 hours without the need to cover or otherwise protect the composition. In some embodiments, it is important for moisture vapor to be able to pass through the film during the time the film is present in order to ensure that the underlying skin is able to breathe and allow sweat to evaporate rather than block skin pores. In some embodiments, the present disclosure provides compositions that have a water vapor transmission rate, as a fraction compared to a non-occluded control, not less than 0.50 over a 48 hour period, not less than 1.0 over a 48 hour period, not less than 1.2 over a 48 hour period the composition, when dried on a porous substrate. In some embodiments, the barrier film of the composition prevents reactions of the skin that are associated with non-breathable topical and transdermal treatments such as intense itching, redness (erythema), and blistering.

High viscosity topical resiquimod or gardiquimod formulations, e.g., creams, lotions and gels, require application to the skin with the palm or fingers of the hand of the user, and thus these types of formulations are associated with a significant risk for secondary resiquimod or gardiquimod exposure. The present claims feature a “sprayable liquid composition” which refer to liquid, i.e., low viscosity compositions, that can be applied directly to the skin in an amount effective via a spray. Such sprayable liquid composition are useful and convenient in delivering the required dosage or resiquimod or gardiquimod needed for the treatment of conditions such as acne, male and female pattern hair loss, hirsutism, hidradenitis suppurativa and/or polycystic ovary syndrome (PCOS). In some embodiments, the “sprayable liquid composition” has low viscosity (about 15 cPs to about 45 cPs) and can be administered in smaller volumes, i.e., in a volume less than 500 μL per actuation. In some embodiments, the sprayable liquid composition has a viscosity suitable for administering via an aerosol spray and/or a mist. For example, in some embodiments, the sprayable liquid composition has a viscosity of less than 200 cPs, less than 100 cPs, less than 50 cPs or less than 30 cPs at room temperature. In some embodiments, the sprayable liquid formation has a viscosity of about 1 cp to about 10 cPs, about 5 cPS to about 20 cPs or about 20 cPs to about 30 cPs at room temperature. In some embodiments, the sprayable liquid composition has a viscosity of about 5 cPs to about 15 cPs. As used throughout the present disclosure, viscosity is measured at standard conditions, e.g., 1 atm pressure and 25° C. temperature.

The sprayable liquid composition of the present disclosure can be administered by means to known in the art. In some embodiments, the disclosure provides a spray container for administering the composition. In some embodiments, the disclosure provides a spray container comprising the sprayable liquid compositions as described herein and a metering valve. In some embodiments, the spray container is a sealed and pressurized device, e.g., an aerosol container. In some embodiments, the spray container is a sealed and pressurized device, an aerosol container, with a means for providing an aerosol spray of the sprayable liquid composition, e.g., an aerosol nozzle. In some embodiments, the spray container further comprises a dose indicator. In some embodiments, the spray container comprises a propellant. In some embodiments, the spray container comprises an amount of the sprayable liquid composition sufficient for a single administration of the composition. In some embodiments, the spray container comprises an amount of the sprayable liquid composition sufficient for two administrations of the composition. In some embodiments, the spray container comprises an amount of the sprayable liquid composition sufficient for three or more administrations of the composition.

Particular embodiments of the present disclosure may include the administration of one or more additional active agents with the administration of resiquimod and/or gardiquimod as described herein. The identity of the additional active agent(s) will be largely dependent on the nature of the underlying condition being treated, e.g., the addition of a moisturizer such as niacinamide may be appropriate in the treatment of skin dryness. Suitable additional active agents include, but are not limited to, other active ingredients readily known to those of skill in the art as useful in the topical treatment of skin disorders or conditions. Exemplary additional active ingredients include, but are not limited to, antibiotics, bactericidal drugs, bacteriostatic drugs, cleansing agents, absorbents, anti-infective agents, anti-inflammatory agents, astringents (drying agents that precipitate protein and shrink and contract the skin), emollients (skin softeners), moisturizers, keratolytics (agents that soften, loosen, and facilitate exfoliation of the squamous cells of the epidermis), and mixtures thereof. In some embodiments, the additional active agent is about 0.1% to about 10% of the sprayable liquid composition. In some embodiments, additional active agent is present in the same sprayable liquid composition as the resiquimod or gardiquimod. In some embodiments, the additional active agent is in a sprayable liquid composition that is distinct from the sprayable liquid composition comprising the resiquimod or gardiquimod. Thus, it is envisioned by the present disclosure that resiquimod or gardiquimod and the additional active agent can be applied from the same dispensing system independently of whether the resiquimod, gardiquimod and the additional active agent are in the same sprayable liquid composition or in distinct sprayable liquid compositions.

Various containers can be used to hold, store or house the compositions disclosed herein in the spray container. In some embodiments, the container can comprise a metal body, preferably lined with a chemically inert coating material to avoid degradation of the composition due to any interaction between the body and the composition. In some embodiments, a container can comprise a plastic body, preferably lined with a chemically inert coating material to avoid degradation of the composition due to any interaction between the body and the composition. In some embodiments, the container is a substantially rigid metal or plastic container adapted to contain a pressurized propellant located within the container and in contact with the product to be dispensed. In some embodiments, the container is an inner substantially rigid metal or plastic container adapted to contain a pressurized propellant located within an outer container made from the same material and away from contact with the product to be dispensed.

In some embodiments, the container body can be constructed from materials such as metal, glass, ceramics, polyester, polyethylene terephthalate (PET) or other polymers. In some embodiments, glass containers can be provided with a safety coating of, for instance, polypropylene to contain glass shards that may be formed on impact with a hard surface. In some embodiments, metal container bodies can be used to withstand impact and are amenable to surface coating. In some embodiments, the container comprises stainless steel, tinplate and aluminum, or combinations thereof. In some embodiments the aluminum is aluminum alloy or anodized aluminum.

Various inert coating materials can be used to line the container body including any suitable coating material known in the art such as a polymer, lacquer, resin or other coating treatment that creates a barrier between the container and the composition for preventing any chemical interaction between the composition and the container. In some embodiments, the inert material is a non-metallic coating. In some embodiments, known coatings for metal containers include acrylic, phenolic, polyester, epoxy and vinyl resins can be used to line the container body disclosed herein. Accordingly, the container coating for use with a composition of the present invention can be selected so that it exhibits no acidic or alkaline reactivity in itself, and that no acidic or alkaline reacting impurities are leached from it in the presence of the composition.

In some embodiments, the interior of a metal container can be lined with materials such as polyamides, polyimides, polypropylene, polyethylene, fluoropolymers, including perfluoroethylenepropylene copolymer (FEP), fluororubber (FPM), ethylene-propylene diene monomer rubber (EPDM), polytetrafluoroethylene (PTFE), ethylene tetrafluoroethylene copolymer (EFTE), perfluoroalkoxyalkanes, perfluoroalkoxyalkylenes, or blends of fluoropolymers with non-fluorocarbon polymers. Fluoropolymers can, for example, be used in combination with polyimide-polyamide resins.

The coating material of the container can be applied as a single layer, or in multiple layers, for example allowing each layer to cure before application of a further layer. The application of more than one coating can be used to shield the composition from the metal container and prevent adhesion of the active ingredients on the container walls.

The term “topical composition” in some embodiments can include the sprayable liquid composition on a surface, e.g., a skin surface, which forms when the aerosol spray comes in contact with the surface. In some embodiments, administration by aerosol spray provides a more consistent administration of the composition on the subject being treated. In some embodiments, administration by aerosol spray provides a more thorough administration of the composition to the subject, e.g., more area is covered. In some embodiments, administration by aerosol spray provides a more convenient method of administration. In some embodiments, administration by aerosol spray reduces the need for other applicators, e.g., wipes, gauzes, clothes, etc. In some embodiments, administration by aerosol spray reduces exposure of the hands or other unintended body parts to the presence of resiquimod or gardiquimod that may occur by administration by creams, gels, or liquids. In some embodiments, administration by aerosol spray provides a more convenient mode of administration, as it allows the composition to dry quickly after application.

In embodiments in which the spray container is a pressurized device, the pressure in the sealed and pressurized device can be any pressure suitable for delivery of the sprayable liquid composition. In some embodiments, the pressure is about 28 psi to about 145 psi at 25° C.

In some embodiments, the spray container comprising the sprayable liquid composition is a misting device, with a means for providing a mist of the topical composition. As used herein, the misting device would include any device which is capable of producing fine mist particles of the sprayable liquid composition. In some embodiments, the misting device produces mist particles of the sprayable liquid composition with an average diameter of about 20 μm to about 150 μm.

### Methods of Treatment

The sprayable liquid compositions described herein can be used in the treatment of arboviral infections in a subject. In some embodiments, the arboviral infection is Dengue, Japanese Encephalitis, yellow fever, kyasanus forest disease, California Encephalitis, Chikungunya, Dengue, Eastern Equine Encephalitis, Powassan, St. Louis Encephalitis, West Nile, and Zika virus disease. In some embodiments, the subject is a human. In some embodiments, the human is a female. In some embodiments, the human is a child. In some embodiments, the human is a male. The present disclosure provides a sprayable liquid composition of resiquimod or gardiquimod that can provide fewer adverse events and can be well suited for both female, male and children subjects who currently have fewer options regarding the management, prevention and treatment of arboviral infections.

The term “treatment” as used herein refers to reducing the severity of a symptom associated with an arboviral infection. Thus, in some embodiments, methods provided herein provide palliative care for arboviral infections. In some embodiments, the term “treatment” refers to eliminating a symptom associated with arboviral infections. Thus, in some embodiments, the methods provided herein provide curative care associated with arboviral infections. In some embodiments, the methods described herein reduce and/or eliminate the severity one, two, three or more than three symptoms associated with arboviral infections.

Interferons (IFNs), the body's first line of antiviral defense, are cytokines that are secreted by host cells in response to virus infection. By inducing the expression of hundreds of IFN-stimulated genes, several of which have antiviral functions, IFNs block virus replication at many levels. Toll-like receptors (TLR) are a family of highly conserved pattern recognition receptors involved in innate immune responses to pathogen infection. Some of the more than two dozen members of this class of receptors that include TLR3, TLR7, TLR8, and TLR9 are localized within intracellular vesicles including the endoplasmic reticulum, endosomes, lysosomes, and endolysosomes. These intracellular TLRs recognize microbial nucleic acids, and once activated, induce rapid antiviral responses characterized by the production of innate immune factors including inflammatory cytokines and antiviral factors. TLR7 and TLR8 were originally identified by the ability to recognize imidazoquinolinone derivatives such as resiquimod, and gardiquimod that possess antiviral and antitumor properties. TLR7 and TLR8 recognize single-stranded RNA from viruses. Overall, imidazoquinolinone derivatives have a unique mode of action that does not directly kill the infected cells, but enhances the production of many pro-inflammatory cytokines and to trigger an immune response.

In some embodiments, the disclosure provides a method of increasing interferon levels in a subject in need thereof the method comprising any one of the compositions disclosed herein as a spray to a skin surface of the subject. In some embodiments, the interferons are toll like receptors (TLRs). In some embodiments, the TLR is TLR3, TLR7, TLR8, and TLR9.

The term “subsequent” as used herein refers to applications of the sprayable liquid compositions that follow or come after another application in time, order, or place. For example, an application of the sprayable liquid composition that is 24 hours after the first application of the composition to the skin is a subsequent application.

In some embodiments, the subsequent application is applied one hour to one week after the first application. In some embodiments, the subsequent application is applied two hours to 4 days after the first application. In some embodiments, the subsequent application is applied four hours to two days after the first application. In some embodiments, the subsequent application is applied six hours to one day after the first application. In some embodiments, the subsequent application is applied twelve hours to one day after the first application. In some embodiments, the subsequent application is applied “as needed” or upon the occurrence of a symptom associated with acne, male and female pattern hair loss, hirsutism, hidradenitis suppurativa, PCOS, or combinations thereof.

In some embodiments, the first application and the second application are applied at the same site on the skin surface of the subject. In some embodiments, the second application of the composition is applied at a site on the skin that is the site of the first application. In some embodiments, the barrier film formed on the skin from the first application of the composition is removed from the first site before the second application of the composition. In some embodiments, the barrier film formed from the first application of the composition is removed from the first site before the composition is applied to the first site in a subsequent application.

In some embodiments, the first application of the composition is applied at a site on the skin that is different from the second site. In some embodiments, the first application and the second application are applied at different sites on the skin surface of the subject.

To achieve a dosage amount sufficient for the intended therapeutic effect, it is often necessary to apply large amounts of resiquimod, or gardiquimod compositions to large surface areas of skin. However, administration of large volumes of a drug increases the likelihood that a solution does not dry in time, and then drips in runoff from the application site, which can result in an inconsistent and inaccurate amounts delivered. Additionally, drug runoff increases the risk of transfer to other persons and unwanted side effects. Achieving ideal dosing concentrations and volumes is critical to achieving consistent and accurate dosage amount in a patient and preventing transfer to others. In some embodiments, the present disclosure provides a composition for which the required volume of composition administered is reduced, and provides a film forming layer which reduces transference.

The sprayable liquid compositions described herein can be applied in a volume of about 500 μL or less in a single actuation. In some embodiments, the composition is applied in a volume of about 300 μL or less in a single actuation. In some embodiments, the composition is applied in a volume of about 250 μL or less in a single actuation. In some embodiments, the composition is applied in a volume of about 100 μL or less in a single actuation. In some embodiments, the composition is applied in a volume of about 50 μL to about 300 μL to the skin in a single actuation. In some embodiments, the composition is applied in a volume of about 100 μL in a single actuation. In some embodiments, the composition is applied in a volume of about 50 μL in a single actuation.

The sprayable liquid compositions provided herein form a protective, washable and breathable film and thus, does not need to be covered after application to the skin. In some embodiments, the sprayable liquid composition is applied to exposed parts of the body, e.g., the arms. In some embodiments, the sprayable liquid composition is applied to an area of the skin that does need to be covered with clothing or occlusive items such as bandages.

In some embodiments, the present disclosure provides for sprayable liquid compositions that require a reduced frequency of administrations for treatment of conditions described herein. For example, in some embodiments, the composition is administered not more than once in a 24 hour period to alleviate symptoms of the conditions described herein.

All references cited herein, including patents, patent applications, papers, textbooks and the like, and the references cited therein, to the extent that they are not already, are hereby incorporated herein by reference in their entirety.

### EXAMPLES

### Example 1—Topical Compositions of Resiquimod and/or Gardiquimod

Various topical compositions comprising resiquimod and/or gardiquimod were prepared according to a manufacturing process described herein. To prepare the compositions, ethanol, and isopropyl alcohol were mixed in a suitable vessel to produce a solvent mixture. The film forming excipients, methacrylic acid and methyl methacrylate copolymer 1:1, methacrylic acid and methyl methacrylate copolymer 1:2, poly(butylmethacrylate-co-(2-dimethylaminoethyl) methacrylate-co-methyl methacrylate 1:2:1, hypromellose, hydroxypropyl cellulose, or ethyl cellulose, and polyethylene glycol 400 were added to the solvent mixture, and the mixture was stirred until a clear solution formed. The penetration enhancers, 1-dodecylazacycloheptan-2-one, isopropyl myristate, octisalate, oleic acid, diethylene glycol monoethyl ether (Transcutol® P), and/or a fragrance were then added to the mixture and the mixture was further stirred. The active ingredients resiquimod and/or gardiquimod were then added to the mixture and the mixture was again stirred until a clear solution was achieved. Lastly, the solution was dispensed into a suitable container and a dispensing pump was affixed to the container. Exemplary resiquimod and/or gardiquimod spray compositions are shown below.

TABLE 1

| | Composition | | | | | | |
|---|---|---|---|---|---|---|---|
| | 1 | 2 | 3 | 4 | 5 | 6 | 7 |
| Ingredient | % w/v | | | | | | |
| Resiquimod | 0.01 | 0.03 | 0.05 | 1.0 | 2.0 | 3.0 | 5.0 |
| and/or | | | | | | | |
| Gardiquimod | | | | | | | |
| Ethanol USP | 61.0 | 58.0 | 56.9 | 60.0 | 70.0 | 68.0 | 72.1 |
| Isopropyl | 30.0 | 29.5 | 27.4 | 25.5 | 15.5 | 13.0 | 10.5 |
| Alcohol USP | | | | | | | |
| Octisalate USP | 4.0 | 6.0 | 7.0 | 5.0 | 6.0 | 7.0 | 6.0 |
| Eudragit ® | 3.0 | 5.0 | 7.0 | 7.0 | 5.0 | 7.0 | 5.0 |
| S 100 USP/NF | | | | | | | |
| Polyethylene | 1.5 | 1.0 | 1.2 | 1.5 | 1.0 | 1.5 | 0.9 |
| glycol 400 | | | | | | | |
| Fragrance | 0.5 | 0.5 | 0.5 | 0.0 | 0.5 | 0.5 | 0.5 |
| TOTAL | 100.0 | 100.0 | 100.0 | 100.0 | 100.0 | 100.0 | 100.0 |

TABLE 2

| | Composition | | | | | | |
|---|---|---|---|---|---|---|---|
| | 8 | 9 | 10 | 11 | 12 | 13 | 14 |
| Ingredient | | | | % w/v | | | |
| Resiquimod | 0.01 | 0.5 | 0.1 | 0.5 | 1.0 | 3.00 | 5.00 |
| and/or | | | | | | | |
| Gardiquimod | | | | | | | |
| Ethanol USP | 58.99 | 63.9 | 65.8 | 70.0 | 72.7 | 41.5 | 67.0 |
| Isopropyl | 22..5 | 19.1 | 17.4 | 10.5 | 9.8 | 41.5 | 10.5 |
| Alcohol | | | | | | | |
| USP | | | | | | | |
| Dimethyl- | 10.0 | 4.0 | 1.0 | 5.0 | 4.0 | 1.0 | 5.0 |
| sulphoxide | | | | | | | |
| USP | | | | | | | |
| Transcutol ® | 4.0 | 6.0 | 7.0 | 5.0 | 6.0 | 4.0 | 6.0 |
| P USP | | | | | | | |
| Eudragit ® | 3.0 | 5.0 | 7.0 | 7.0 | 5.0 | 7.0 | 5.0 |
| E100 | | | | | | | |
| USP/NF | | | | | | | |
| Polyethylene | 1.5 | 1.0 | 1.2 | 1.5 | 1.0 | 1.5 | 0.9 |
| glycol 400 | | | | | | | |
| Fragrance | 0.0 | 0.5 | 0.5 | 0.5 | 0.5 | 0.5 | 0.6 |
| TOTAL | 100.0 | 100.0 | 100.0 | 100.0 | 100.0 | 100.0 | 100.0 |

TABLE 3

| | Composition | | | | | | |
|---|---|---|---|---|---|---|---|
| | 15 | 16 | 17 | 18 | 19 | 20 | 21 |
| Ingredient | % w/v | | | | | | |
| Resiquimod | 1.5 | 3.0 | 6.0 | 7.0 | 9.00 | 10.00 | 25.00 |
| and/or | | | | | | | |
| Gardiquimod | | | | | | | |
| Ethanol USP | 46.5 | 50.5 | 48.9 | 52.5 | 56.5 | 55.0 | 27.0 |
| Isopropyl | 28.5 | 26.5 | 21.4 | 16.5 | 13.5 | 11.0 | 25.5 |
| Alcohol | | | | | | | |
| USP | | | | | | | |
| Oleic Acid | 15.0 | 15.0 | 15.0 | 15.0 | 15.0 | 15.0 | 13.0 |
| USP | | | | | | | |
| Eudragit ® | 7.0 | 4.0 | 7.0 | 7.0 | 5.0 | 7.0 | 7.0 |
| E100 USP/NF | | | | | | | |
| Polyethylene | 1.5 | 1.0 | 1.2 | 1.5 | 1.0 | 1.5 | 2.0 |
| glycol 400 | | | | | | | |
| Fragrance | 0.5 | 0.0 | 0.5 | 0.5 | 0.0 | 0.5 | 0.5 |
| TOTAL | 100.0 | 100.0 | 100.0 | 100.0 | 100.0 | 100.0 | 100.0 |

TABLE 4

| | Composition | | | | | | |
|---|---|---|---|---|---|---|---|
| | 22 | 23 | 24 | 25 | 26 | 27 | 28 |
| Ingredient | % w/v | | | | | | |
| Resiquimod | 0.9 | 0.9 | 1.8 | 2.6 | 3.0 | 4.5 | 7.5 |
| and/or | | | | | | | |
| Gardiquimod | | | | | | | |
| Ethanol USP | 58.7 | 58.8 | 55.4 | 61.1 | 62.7 | 58.9 | 61.9 |
| Isopropyl | 28.2 | 28.2 | 27.3 | 22.8 | 22.2 | 21.2 | 18.0 |
| Alcohol | | | | | | | |
| USP | | | | | | | |
| Octisalate USP | 4.0 | 6.0 | 7.0 | 5.0 | 6.0 | 7.0 | 6.0 |
| Eudragit ® | 7.0 | 5.0 | 7.0 | 7.0 | 5.0 | 7.0 | 5.5 |
| S100 USP/NF | | | | | | | |
| Polyethylene | 1.2 | 1.1 | 1.5 | 1.5 | 1.1 | 1.4 | 1.1 |
| Glycol 300 | | | | | | | |
| TOTAL | 100.0 | 100.0 | 100.0 | 100.0 | 100.0 | 100.0 | 100.0 |

TABLE 5

| | Composition | | | | | |
|---|---|---|---|---|---|---|
| | 29 | 30 | 31 | 32 | 33 | 34 |
| Ingredient | % w/v | | | | | |
| Resiquimod | 0.6 | 0.6 | 0.6 | 1.2 | 1.2 | 1.2 |
| and/or | | | | | | |
| Gardiquimod | | | | | | |
| Ethanol USP | 58.7 | 58.9 | 55.5 | 61.3 | 62.9 | 59.4 |
| Isopropyl | 28.5 | 28.5 | 28.5 | 24.0 | 24.0 | 24.0 |
| Alcohol USP | | | | | | |
| Octisalate USP | 4.0 | 6.0 | 7.0 | 5.0 | 6.0 | 7.0 |
| Eudragit ® | 7.0 | 5.0 | 7.0 | 7.0 | 5.0 | 7.0 |
| E100 USP/NF | | | | | | |
| Polyethylene | 1.2 | 1.0 | 1.4 | 1.5 | 0.9 | 1.4 |
| Glycol 300 | | | | | | |
| TOTAL | 100.0 | 100.0 | 100.0 | 100.0 | 100.0 | 100.0 |

TABLE 6

| | Composition | | | | | |
|---|---|---|---|---|---|---|
| | 35 | 36 | 37 | 38 | 39 | 40 |
| Ingredient | % w/v | | | | | |
| Resiquimod and/or | 2.25 | 2.25 | 2.25 | 3.0 | 3.0 | 3.0 |
| Gardiquimod | | | | | | |
| Ethanol USP | 58.8 | 58.9 | 54.4 | 61.3 | 62.9 | 59.4 |
| Isopropyl | 26.75 | 26.85 | 26.95 | 22.2 | 22.2 | 22.2 |
| Alcohol USP | | | | | | |
| Octisalate USP | 4.0 | 6.0 | 7.0 | 5.0 | 6.0 | 7.0 |
| Eudragit ® | 7.0 | 5.0 | 7.0 | 7.0 | 5.0 | 7.0 |
| S100 USP/NF | | | | | | |
| Polyethylene | 1.2 | 1.0 | 1.4 | 1.5 | 0.9 | 1.4 |
| Glycol 300 | | | | | | |
| TOTAL | 100.0 | 100.0 | 100.0 | 100.0 | 100.0 | 100.0 |

### Example 2—Analysis of Skin Permeation of Resiquimod and Gardiquimod Film Forming Compositions

The degree of permeation of resiquimod and gardiquimod from a film forming composition into skin was tested. Franz diffusion cells with a donor area of 4.9 cm2 and 20 mL receptor chamber volume were assembled with excised abdominal porcine skin. The receptor chamber was filled with phosphate buffered saline (PBS) at pH 7.4. For the experiment, 60 μl of 0.01% w/v of resiquimod and gardiquimod composition was added to the donor chamber of the cell. (Composition 1). The samples were maintained at 37° C. and the receptor chamber solution was magnetically stirred to maintain homogeneity. Samples of receptor medium were collected at 1, 2, 4, 6, and 24 hour timepoints post addition of the resiquimod and gardiquimod composition to the cells and then analyzed by HPLC to determine the amount of drug permeated through the skin layers.

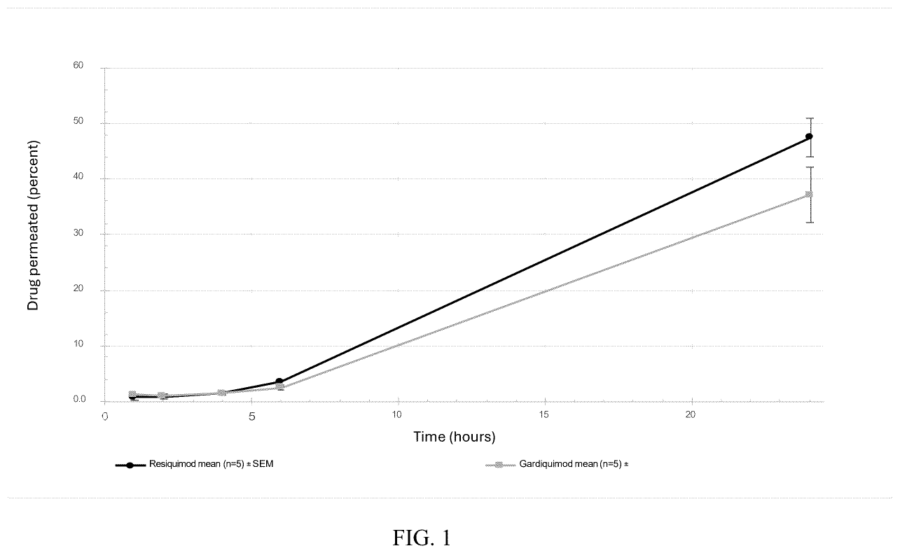

As shown in FIG. 1, the active agents permeated into the skin rapidly for the 0.01% w/v of resiquimod and gardiquimod film forming composition. The profiles show the skin acts as a reservoir from which resiquimod and gardiquimod permeated from the skin over 24 hours.

### Example 6 Analysis of Crystallization in Film Forming Composition

The film forming composition was evaluated for any crystallization of resiquimod and gardiquimod. The compositions were prepared according to the manufacturing process as described herein. The compositions were then analyzed visually under the microscope for evidence of resiquimod or gardiquimod crystallization.

| Composition | Observation |
|---|---|
| 1.0% w/v resiquimod or gardiquimod and 7% | No crystals formed |
| w/v film forming composition | |

1. A topical composition comprising
a. about 0.01% w/v to about 5% w/v of resiquimod, gardiquimod and/or derivatives thereof;
b. a penetration enhancer;
c. at least 45% w/v of an aliphatic solvent or a combination thereof; and
d. a film forming excipient,
wherein the film forming excipient has a solubility in water, at a pH between 1 and 10, wherein the composition is a sprayable liquid solution that forms a washable film when sprayed on skin, and wherein the resiquimod, gardiquimod and/or derivatives thereof do not crystallize when the composition is applied to the skin surface.

2-5. (canceled)

6. The composition of claim 1, wherein the penetration enhancer comprises 1-dodecylazacycloheptan-2-one, isopropyl myristate, octisalate, oleic acid, diethylene glycol monoethyl ether (Transcutol® P), or combinations thereof.

7. (canceled)

8. (canceled)

9. The composition of claim 1, wherein the solvent comprises acetone, di-isopropyl adipate, dimethyl isosorbide, dimethyl sulfoxide, ethyl acetate, ethanol, isopropyl alcohol or combinations thereof.

10. (canceled)

11. The composition of claim 1, wherein the film forming excipient comprises a polyacrylate polymer or a cellulose polymer.

12. The composition of claim 1, wherein the film forming excipient is methacrylic acid and methyl methacrylate copolymer 1:1, methacrylic acid and methyl methacrylate copolymer 1:2, poly (butylmethacrylate-co-(2-dimethylaminoethyl) methacrylate-co-methyl methacrylate 1:2:1, hypromellose, hydroxypropyl cellulose, and ethyl cellulose.

13. (canceled)

14. (canceled)

15. The composition of claim 1, wherein the composition further comprises a washability enhancer.

16. The composition of claim 15, wherein the washability enhancer is polyethylene glycol 400.

17. (canceled)

18. The composition of claim 1, wherein the pH of the composition is about 4 to about 5.

19. (canceled)

20. The composition of claim 1, wherein the composition forms a barrier film in less than five minutes after application to the skin.

21. (canceled)

22. (canceled)

23. The composition of claim 20, wherein the barrier film formed is water washable.

24. The composition of claim 1, wherein the composition has a viscosity of less than 30 cPs at room temperature.

25. The composition of claim 1, wherein the composition has a water vapor transmission rate, as a fraction compared to a non-occluded control, not less than 0.50 over a 48-hour period when applied and dried on a porous substrate.

26. The composition of claim 20, wherein the barrier film is capable of preventing transfer of resiquimod, gardiquimod and/or derivatives thereof to another human.

27. A spray container comprising
a. the composition of claim 1; and
b. a metering valve.

28. The spray container of claim 27, wherein the container further comprises an applicator for spreading the composition onto human skin.

29. (canceled)

30. (canceled)

31. The spray container of claim 27, wherein the container further comprises a propellent.

32. A method of treating an arboviral infection, in a subject in need thereof, the method comprising topically applying the composition of claim 1 as a spray to a skin surface of the subject, wherein the arboviral infection is Dengue, Japanese Encephalitis, Yellow fever, kyasanus forest disease, California Encephalitis, Chikungunya, Dengue, Eastern Equine Encephalitis, Powassan, St. Louis Encephalitis, West Nile, Zika virus disease, or combinations thereof.

33. (canceled)

34. A method of increasing interferon levels in a subject in need thereof the method comprising topically applying the composition of claim 1.

35. The method of claim 34, wherein the interferons are toll like receptors (TLR).

36-45. (canceled)

46. The method of claim 32, wherein the composition is applied to the skin surface in a single actuation in a volume of about 500 μL or less.

47-49. (canceled)
